(19) United States
(12) Patent Application Publication
(10) Pub. No.: US 2009/0191634 A1
(43) Pub. Date: Jul. 30, 2009

(54) (METH)ACRYLATE SURFACES FOR CELL CULTURE, METHODS OF MAKING AND USING THE SURFACES

(76) Inventors: Arthur W Martin, Horseheads, NY (US); Zara Melkoumian, Painted Post, NY (US); Christopher B. Shogbon, Corning, NY (US); Yue Zhou, Horseheads, NY (US)

Correspondence Address: CORNING INCORPORATED, SP-TI-3-1, CORNING, NY, 14831, US

(21) Appl. No.: 12/362,756

(22) Filed: Jan. 30, 2009

Related U.S. Application Data
(60) Provisional application No. 61/062,888, filed on Jan. 30, 2008.

(51) Int. Cl. C12N 5/00; C08F 126/02; C08F 224/00; C08F 220/26; C08F 26/06; C08J 7/18; C08F 226/10; C08F 120/56; C08F 20/10

(52) U.S. Cl. 435/402; 526/261; 526/266; 526/320; 526/260; 526/310; 526/258; 526/303.1; 526/328; 427/508

(57) ABSTRACT

A synthetic cell culture surface, prepared from a polymerized blend of at least two (meth)acrylate monomers is provided, which supports the growth of undifferentiated human embryonic stem cells in defined media augmented with fetal bovine serum. The cell culture surface forms a uniform layer over the growth area of a typical cell culture vessel.

### CROSS REFERENCE TO RELATED APPLICATION

This Application claims the benefit of U.S. Provisional Application Ser. No. 61/062,888 filed Jan. 30, 2008 and entitled “(meth)acrylate Surfaces for Cell Culture, Methods of Making and Methods of Using the Surfaces”.

### FIELD

The present invention relates generally to surfaces and surface treatments to promote cell culture. More specifically, the present invention relates to (meth)acrylate compounds and combinations of (meth)acrylate compounds as cell culture surfaces. The present invention also provides methods of making and methods of using the cell culture surfaces.

### BACKGROUND

In vitro culturing of cells has been a useful research tool, providing material necessary for research in pharmacology, physiology and toxicology. Recent advances in the field of developmental biology, significantly in the isolation, growth and differentiation of stem cells, have opened the door for cell culture to provide material for therapeutic applications as well. Embryonic stem cells, specifically human embryonic stem cells, may be able to provide answers to difficult medical problems such as Alzheimer's disease, Parkinson's disease, diabetes, spinal cord injury, heart disease, and other debilitating and often fatal conditions.

However, embryonic stem cells are difficult to culture, difficult to control, and often require a specialized cell culture surface that can facilitate growth and proliferation in the undifferentiated state. Many coatings and surface enhancements have been developed to provide cell culture surfaces which promote cell growth in vitro. Many of these surfaces contain animal-derived additives such as proteins or cell extracts. These additives introduce a risk of infection into the preparation of the therapeutic cells. For example, the use of extra-cellular matrix proteins derived from animals may introduce infective agents such as viruses or prions. These infective agents may be taken up by cells in culture and, upon the transplantation of these cells into a patient, may be taken up into the patient. Therefore, the addition of these factors in or on cell culture surfaces may introduce new disease even as they address an existing condition. In addition, these animal-derived additives or cell surface coatings may lead to significant manufacturing expense and lot-to-lot variability which are not preferable. There is a need for cell culture surfaces which do not include animal-derived ingredients or additives and which provide cell culture conditions amenable for the culture of difficult-to-culture cells including embryonic stem cells.

### SUMMARY

Embodiments of the present invention provide (meth)acrylate compounds and combinations of (meth)acrylate compounds as surfaces for cell culture. In embodiments, the cell culture surface forms a uniform layer over the growth area of a typical cell culture vessel. In embodiments, the invention provides a composition for making cell culture surfaces having a blend of at least two UV-curable (meth)acrylate monomers where one of the at least two (meth)acrylate monomers is selected from the group consisting of tris(2-hydroxy-ethyl) isocyanurate triacrylate, tetrahydrofurfuryl acrylate, proxylated triglycerol triacrylate, 2-N-morpholinoethyl methacrylate, bis(2-methacryoyloxyethyl) N,N′-1,9-nonylene biscarmate diurethane dimethacrylate, 2-(2-oxo-1-imidazolidinyl) methacrylate 1-vinyl imidazole, N-vinyl-2-pyrrolidone methacrylate, pentaerythritol triacrylate, N—N-dimethyl acrylamide, stearyl acrylate, lauryl acrylate, lauryl methacrylate, dicyclopentadienyl methacrylate, caprolactone acrylate, and 2(2-ethoxyethoxy) ethylacrylate, dipentaerythritol penta-acrylate, 2 (dimethyl amino) ethyl methacrylate, pentaerythritol tri-acrylate, and 2-(t-butylamino)ethyl methacrylate.

In additional embodiments, the present invention provides compositions where an additional UV-curable acrylate monomer is 1,6 hexanediol diacrylate, tetraethylene glycol dimethacrylate, Tripropyleneglycol Diacrylate, 1,4-Butanediol Diacrylate Trimethylpropane Triacrylate or 1,5 pentanediol dimethacrylate. In embodiments, the composition for making cell culture surfaces includes a solvent which may be a volatile solvent and may be ethanol.

In further embodiments, the present invention provides compositions for making cell culture surfaces where at least five percent of the curable (meth)acrylate monomers mixed together to make the surface are cross-linking monomers. In additional embodiments, the present invention provides compositions for making cell culture surfaces having a blend of at least two UV-curable (meth)acrylate monomers and a solvent where the solvent is not dimethylformamide (DMF), dichloromethane (DCM) or tetrahydrofuran (THF). In further embodiments, the present invention provides a cell culture article comprising a polymeric substrate, a surface for cell culture on the polymeric substrate comprising a polymeric blend of at least two (meth)acrylate monomers where the resulting surface for cell culture is larger in diameter than 1000 μm, or where the resulting surface has a contact angle of less than 80° and a modulus of from 1000 to 5500 mPa.

In additional embodiments, the present invention provides a method for preparing a cell culture surface with the following steps: mixing at least two (meth)acrylate monomers together with a photopolymerizing agent in a solvent wherein the solvent is not DMF, DCM or THF; applying the at least two (meth)acrylate monomer mixture to a cell culture substrate; allowing the solvent to evaporate; and, exposing the coated substrate to UV light.

In embodiments, the present invention provides a mixture of monomers for making a cell culture surface where the mixture of monomers is tris(2-hydroxy-ethyl) isocyanurate triacrylate, 1,6 hexanediol diacrylate and trimethylpropane triacrylate; tetrahydrofurfuryl acrylate, N-vinyl-2-pyrrolidone methacrylate, and N—N-dimethyl acrylamide; proxylated triglycerol triacrylate, N-vinyl-2-pyrrolidone methacrylate and tripropylene glycol diacrylate; morpholinoethyl methacrylate and tripropylene glycol diacryate; bis(2-methacryoyloxyethyl) N,N′-1,9-nonylene biscarmate diurethane dimethacrylate, 1,4-butanediol diacrylate and N-Hexyl acrylate; bis(2-methacryoyloxyethyl) N,N′-1,9-nonylene biscarmate diurethane dimethacrylate, 1,4-butanediol diacrylate, N-Hexyl acrylate and 2-N-morpholinoethyl methacrylate; bis(2-methacryoyloxyethyl) N,N′-1,9-nonylene biscarmate diurethane dimethacrylate, 1,4-butanediol diacrylate, N-Hexyl acrylate and 1-vinyl imidazole; bis(2-methacryoyloxyethyl) N,N′-1,9-nonylene biscarmate diurethane dimethacrylate, 1,4-butanediol diacrylate and N-Hexyl acrylate, tripropylene glycol diacrylate and 1,5-pentanediol dimethacrylate; 2-(2-oxo-1-imidazolidinyl) methacryalte, tetraethylene glycol dimethacrylate and 1,4-butanediol dimethacrylate; polypropylene glycol (400) dimethacrylate, tetraethylene glycol dimethacrylate and 1,4-butanediol dimethacrylate; 2-dimethyl amino ethyl methacrylate, tetraethylene glycol dimethacrylate and 1,4-butanediol dimethacrylate; Pentaerythritol triacrylate, tetraethylene glycol dimethacrylate and 1,4-butanediol dimethacrylate; Caprolactone acrylate, tetraethylene glycol dimethacrylate and 1,4-butanediol dimethacrylate; tetrahydrofurfuryl acrylate, tripropylene glycol diacrylate, and 1,5-pentanediol dimethacrylate; 2-(2-ethoxyethoxyl)ethyl acrylate, tripropylene glycol diacrylate, and 1,5-pentanediol dimethacrylate; lauryl methacrylate, tripropylene glycol diacrylate, and 1,5-pentanediol dimethacrylate; N—N-dimethyl acrylamide, 2-(2-oxo-1-imidazolidinyl) methacrylate, N-vinyl-2-pyrrolidone methacrylate, and pentaerythritol triacrylate; or, Dimethyl acrylamide, pyrrolidone methacrylate, 2(2-ethoxyethoxy) methacrylate; pentaerythritol triacrylate; and, Tripropylene glycol dimethacrylate and dipenta-erythritol penta-acrylate.

In embodiments, the present invention also provides a cell culture system having a cell culture vessel having a cell culture surface comprising a blend of at least two (meth)acrylate monomers, culture media with at least 20% fetal bovine serum and human embryonic stem cells, where the human embryonic stem cells may be differentiated or undifferentiated.

### BRIEF DESCRIPTION OF THE DRAWINGS

FIG. 1 is a graph illustrating contact angles for embodiments of the (meth)acrylate-coated surfaces of the present invention.

FIG. 2 is a graph illustrating zeta potential or surface charge for embodiments of the (meth)acrylate-coated surfaces of the present invention.

FIG. 3 is a graph illustrating modulus for embodiments of (meth)acrylate-coated surfaces of the present invention.

FIG. 4 is a three-dimensional plot of surfaces showing contact angle, surface charge, and modulus.

FIG. 5 is a graph illustrating fluorescence intensities, measuring undifferentiated cell growth on selected embodiments of (meth)acrylate-coated surfaces of the present invention, normalized to fluorescence intensities measuring undifferentiated cell growth on Matrigel™.

FIG. 6 shows a 96 well plate where the growth surfaces on the bottom of the wells of the 96 well plate have been coated with an embodiment of the (meth)acrylate cell culture surface of the present invention.

FIG. 7 is a pair of micrographs, at different magnifications, illustrating BCIP stained cell growth and colony morphology for hES cells grown on Matrigel™.

FIG. 8 is a pair of micrographs, at different magnifications, illustrating BPIC stained cell growth and colony morphology for hES cells grown on embodiments of (meth)acrylate-coated surfaces of the present invention.

FIG. 9 is a graph illustrating AttoPhos measurements for hES cells grown on embodiments of (meth)acrylate-coated surfaces of the present invention.

FIG. 10 is a graph illustrating AttoPhos measurements for hES cells grown on additional embodiments of (meth)acrylate-coated surfaces of the present invention.

### DETAILED DESCRIPTION

Embodiments of the present invention include (meth)acrylate monomers or combinations of (meth)acrylate monomers which provide cell culture surfaces suitable for culturing cells including difficult-to-culture cells such as embryonic stem cells. Embryonic stem cells (ESCs), including human embryonic stem cells (hESCs), are able to grow and self-renew unlimitedly; they can be propagated in culture for extended periods and have an ability to differentiate to multiple cell types. However, these cells have specific cell culture needs. Slight changes in culture conditions can cause these cells to differentiate, or exhibit reduced growth and propagation characteristics. In many cases, hESC cultures require the addition of animal-derived materials either in or on a cell culture surface to effectively grow in culture. These animal-derived materials may harbor pathogens such as infective proteins and viruses, including retroviruses. Although some substrates have demonstrated the ability to facilitate proliferation of hESC in both undifferentiated (pluripotent) and differentiated states, they may still be considered inadequate for cell cultures that are directed toward the development of cell therapeutics in humans because of the threat of pathogens that might be carried from an animal source of cell culture additives to the cultured cells, to an individual treated with those cells. In addition, these animal-derived surfaces may have high lot-to-lot variability making results less reproducible, and they may be very expensive. In light of these disadvantages, surfaces that include animal-derived materials may be relegated to academic and pre-clinical research and may not be useful to produce, for example, stem cells to treat patients. Furthermore, because of the costs associated with these animal derived surfaces, they are considered very expensive even for academic research, leaving the door open for cheaper and safer alternatives. Therefore, to provide a product which eliminates the risks associated with animal derived products, synthetic (meth)acrylate surfaces with special surface attributes are proposed.

Preferable cell culture surfaces may be made from ingredients which are not animal-derived, may sustain at least 15 passages of cells in cell culture, may be reliable and reproducible, and may allow for the growth of cells which show normal characteristics, normal karyotype, after defined passages. Preferable cell culture surfaces for stem cells may be made from ingredients which are not animal-derived, and sustain undifferentiated growth of ES cells for at least 10 passages in culture. Preferable cell culture surfaces may also be stable. Cell culture surfaces may be non-toxic. They may be able to withstand processing conditions including sterilization, possess adequate shelf life, and maintain quality and function after normal treatment. In addition, preferable cell culture surfaces may be suitable for large-scale industrial production. They may be scalable and cost effective to produce. The materials may also possess chemical compatibility with aqueous solutions and physiological conditions found in cell culture environments.

Cell culture studies conducted on synthetic surfaces have demonstrated that surface properties of substrates can affect the success of cell culture and can affect characteristics of cells grown in culture. For example, surface properties can elicit cell adhesion, spreading, growth and differentiation of cells. Research conducted with human fibroblast cells 3T3 and HT-1080 fibrosarcoma cells has shown correlation with surface energetics, contact angle, surface charge and modulus (Altankov, G., Richau, K., Groth, T., The role of surface zeta potential and substratum chemistry for regulation of dermal fibroblasts interaction, Mat.-wiss. U. Werkstofftech. 2003, 34, 12, 1120-1128.) Anderson et al (2005/0019747) disclosed depositing microspots of (meth)acrylates, including polyethylene glycol (meth)acrylates, onto a substrate as surfaces for stem cell-based assays and analysis. Self-Assembled Mono-layers (SAMS) surfaces with covalently linked laminin adhesive peptides have been used to enable adhesion and short-term growth of undifferentiated hES cells (Derda, S., Li, Lingyin, Orner, B. P., Lewis, R. L., Thomson, A. J., Kiessling, L. L., Defined Substrates for Human Embryonic Stem Cell Growth Identified from surface Arrays, ACS Chemical Biology, Vol. 2, No. 5, May 2, 2007, pp 347-355.

In embodiments of the present invention, polymeric surfaces composed of cross-linked blends of (meth)acrylate monomers that impart specific physical and chemical attributes to the surface are provided. These specific physical and chemical attributes may facilitate the proliferation of undifferentiated hESCs in embodiments of the present invention. These (meth)acrylate surfaces contain monomers with different properties. The monomers have particular characteristics which, when combined and cross-linked, provide (meth)acrylate surfaces that are amenable for cell culture. These characteristics may include hydrophilicity or hydrophobicity, positive charge, negative charge or no charge, and compliant or rigid surfaces. For example, monomers or combinations of monomers which are hydrophilic may provide cell culture surfaces that are preferable in embodiments of the present invention. Or, monomers or combinations of monomers which carry a charge may be preferable in embodiments of the present invention. Or, monomers or combinations of monomers which fall within a certain range of modulus or hardness may be preferable in embodiments of the present invention. Or, monomers or combinations of monomers which exhibit a combination of these attributes may be preferable in embodiments of the present invention.

For the purposes of this disclosure, the term “(meth)acrylate” means compounds that are esters which contain vinyl groups, that is, two carbon atoms double bonded to each other, directly attached to a carbonyl carbon. An acrylate moiety is a moiety of the following formula: CH₂CHC(O)O⁻. Some acrylates, methacrylates, have an extra methyl group attached to the α-carbon and these are also included in the term “(meth)acrylate” for the purposes of this disclosure. A methacrylate moiety is a moiety of the following formula: CH₂C(CH₃)C(O)O⁻. “acrylate” and “(meth)acrylate” are used herein interchangeably, except when content clearly dictates otherwise, e.g. when a specific compound or group of compounds are named. “(meth)acrylate” includes compounds which contain single (meth)acrylate groups or multiple (meth)acrylate groups. “(meth)acrylate” includes acrylates and methacrylates as well as polymerized and unpolymerized monomers (oligomers) with varying reactive functionality, that is, dimers, trimers, tetramers or additional polymers containing acrylic or methacrylic acid groups. “UV-curable monomers,” for the purposes of this disclosure means monomers that can be cross-linked to form polymers by exposure to UV light. In addition, for the purposes of this disclosure, the term “UV-curable monomers” includes compounds described in Tables 1-6. These compounds can also possess non-reactive or reactive moieties in their backbones such as amine, carboxyl, urethane, cyanurate, glycol, diol, ring structures such as furan, imidazole, morphilino and pyrrolidone. In additional embodiments, based on the aforementioned chemical moieties, the present invention provides a semi-rational (meth)acrylate library which provides compounds which impart a wide range of surface properties including surface charge, contact angle and modulus to a surface. These compounds may provide an extensive library for screening, with surface properties that either possess a synergistic effect or act independently in providing an amenable environment in mediating growth and proliferation of undifferentiated human embryonic stem cells in serum-supplemented conditions. The semi-rational library of the present invention uses binary (blends of two (meth)acrylates), tertiary, quaternary or more blends of (meth)acrylate monomers to create cell culture surfaces which provide a cell culture environment amenable to the growth and proliferation of undifferentiated stem cells.

In embodiments of the present invention, monomers and combinations of monomers are applied to a cell culture substrate. The cell culture substrate may be any surface known in the cell culture art. For example, substrates may be gas permeable or gas impermeable polymeric substrates or membranes made of suitable materials that may include for example: polystyrene, polyethylene, polyethyleneterephthalate, polyethylene-co-vinyl acetate, nylon, polycarbonate, polyolefin, ethylene vinyl acetate, polypropylene, polysulfone, polytetrafluoroethylene (PTFE) or compatible fluoropolymer, silicone rubber or copolymer, poly(styrene-butadiene-styrene), cyclic olefin copolymer, polymers, copolymers and combinations of these materials. The substrate may be treated to alter the surface characteristics of the substrate in order for surfaces to facilitate sustainable adhesion between thermoplastic substrates and said (meth)acrylate components. For example, the substrate may be plasma treated, chemically treated, heat treated, mechanically etched, or have increased charged chemical groups available at the surface of the polymer substrate in which (meth)acrylate coating is to be applied. The substrate to be coated may not just be polymeric but can also be silica, glass, ceramic, glass-ceramic, metal or other inorganic material surface.

In an embodiment, the substrate may be a plasma-treated polystyrene, polyolefin or cyclic olefin co-polymer surface. The plasma-treated cyclic olefin co-polymer (cyclic norbonene-ethylene) surface may be, for example, that material sold under the name of Topas® by Topas Advanced Polymers, Florence, Ky. In embodiments, the (meth)acrylate cell culture surface or polymer mixture can be applied to a substrate using methods known in the art, including dip coating, spray coating, spin coating, or liquid dispensing.

In embodiments, the substrate may form part of a cell culture article. Cell culture articles are containers suitable for containing cells in culture. Cell culture articles include flasks, bottles, plates, multi-well plates, multi-layer flasks, dishes, cell culture container inserts, beads, fibers, bags, bioreactors, and/or any type of cell culture vessel or container known in the art. While all sizes are contemplated, in embodiments of the present invention, the (meth)acrylate cell culture surface covers a surface of the cell culture article that is larger than a small spot, or microspot, or larger than 1000 μm in diameter, in the cell culture article. In embodiments, the (meth)acrylate cell culture surface of the present invention covers an entire cell culture surface in the cell culture container or vessel. For example, in embodiments, the (meth)acrylate cell culture surface of the present invention covers the bottom, the cell culture growth surface, of a well of a 96-well plate. Or, in embodiments, the cell culture surface of the present invention covers the cell culture growth surface of a standard cell culture flask. Those of ordinary skill will recognize that embodiments of the present invention may provide cell culture surfaces for known cell culture vessels and containers.

In embodiments of the present invention, the choice of solvent may be important. For example, some solvents such as dichloromethane (DCM) or tetrahydrofuran (THF) might dissolve commonly used substrates such as polystyrene or cyclic olefin copolymers. Or some solvents may not be appropriate for other reasons. For example dimethylformamide (DMF) has a high boiling point which would make it a poor choice for a method requiring the evaporation of a solvent at room temperature.

Because embodiments of the (meth)acrylate cell culture surfaces of the present invention are larger than microspots, and provide surfaces that are consistent with sizes of known cell culture vessels and containers, these cell culture surfaces are useful for culturing cells, including embryonic stem cells (ESCs) and hESCs. In embodiments, the cell culture surfaces of the present invention are useful in providing a cell culture environment amenable to cell culture. In embodiments, the cell culture surfaces of the present invention are useful in providing a cell culture environment amenable to the growth and proliferation of undifferentiated stem cells as well as any other cell type including primary cells, cell lines, tissues and differentiated cells derived from stem cells. Undifferentiated embryonic stem cells are used here as an example of difficult-to-culture cell types.

Cells in culture, including embryonic stem cells and human embryonic stem cells, require medium. Research in the area of synthetic substrates has claimed positive results using medium supplemented with serum replacement and conditioned with mouse embryonic fibroblasts (MEFs) (Li, J. Ying, Chung, E. H., Rodriguez, Firpo, M. T., Healy, K. E., Hydrogels as Artificial matrices for Human Embryonic Stem Cell Self-Renewal, Journal of Biomedical Materials Research part A, Jun. 1, 2006, volume 79A, Issue 1, pp 1-5C). Chemically defined medium, medium in which all components are known is available from a number of vendors including, for example, Stem Cell Technologies, Invitrogen, Carlsbad Calif., and Millipore, Bedford, Mass. In order to facilitate growth of a particular cell type, including undifferentiated hESC cells, as well as differentiation into particular cell types, additives such as growth factors may be added to the chemically defined media. These growth factors may include but are not limited to transforming growth factor-alpha (TGF-alpha), transforming growth factor-beta. (TGF-beta), platelet-derived growth factors including the AA, AB and BB isoforms (PDGF), fibroblast growth factors (FGF), including FGF acidic isoforms 1 and 2, FGF basic form 2, and FGF 4, 8, 9 and 10, hbFGF, nerve growth factors (NGF) including NGF 2.5s, NGF 7.0s and beta NGF and neurotrophins, brain derived neurotrophic factor, cartilage derived factor, bone growth factors (BGF), basic fibroblast growth factor, insulin-like growth factor (IGF), vascular endothelial growth factor (VEGF), EG-VEGF, VEGF-related protein, Bv8, VEGF-E, granulocyte colony stimulating factor (G-CSF), insulin like growth factor (IGF) I and II, hepatocyte growth factor (HGF), glial neurotrophic growth factor (GDNF), stem cell factor (SCF), keratinocyte growth factor (KGF), transforming growth factors (TGF), including TGFs alpha, beta, beta1, beta2, and beta3, skeletal growth factor, bone matrix derived growth factors, and bone derived growth factors and mixtures thereof. Some growth factors can also promote differentiation of a cell or tissue. TGF, for example, can promote growth and/or differentiation of a cell or tissue. Some preferred growth factors include VEGF, NGFs, PDGF-AA, PDGF-BB, PDGF-AB, FGFb, FGFa, hbFGF, HGF, and BGF. Medium may be conditioned, e.g. exposed to a feeder layer of cells, or non-conditioned. In addition, serum may be added to the media. Fetal bovine serum, FBS is available from many sources including Hyclone and Sigma-Aldrich. For the purposes of the experiments described herein, X-Vivo-10 serum-free media from Lonza, Basel, Switzerland was used, amended with the addition of 80 ng/ml hbFGF and 0.5 ng/ml hTGF-β1, and included at least 20% FBS.

Stem cells include adult and embryonic stem cells. Human Embryonic cells in cell lines include CH01, CH02, CY12, CY30, CY40, CY51, CY81, CY82, CY91, CY92, CY10, GE01 (WA01,also known as H1), GE07 (WA07, H7), GE09 (WA09, H9), GE13, GE14, GE91, GE92, SA04-SA19, KA08, KA09, KA40, KA41, KA42, KA43, MB01, MB02, MB03, MI01, NC01, NC02, NC03, RL05, RL07, RL10, RL15, RL20, RL21, as well as numerous others. Stem cells may also be primary cells obtained from embryonic sources, such as surplus in vitro fertilized eggs. Examples of stem cells include, but are not limited to, embryonic stem cells, bone marrow stem cells and umbilical cord stem cells. Induced primate pluripotent stem (iPS) cells may also be used. iPS cells refer to cells, obtained from a juvenile or adult mammal, such as a human, that are genetically modified, e.g., by transfection with one or more appropriate vectors, such that they are reprogrammed to attain the phenotype of a pluripotent stem cell such as an hESC. Phenotypic traits attained by these reprogrammed cells include morphology resembling stem cells isolated from a blastocyst as well as surface antigen expression, gene expression and telomerase activity resembling blastocyst derived embryonic stem cells. The iPS cells typically have the ability to differentiate into at least one cell type from each of the primary germ layers: ectoderm, endoderm and mesoderm and thus are suitable for differentiation a variety of useful cell types. The iPS cells, like hESC, also form teratomas when injected into immuno-deficient mice, e.g., SCID mice. (Takahashi et al., (2007) Cell 131(5):861; Yu et al., (2007) Science318:5858). Other examples of cells used in various embodiments include, but are not limited to, myoblasts, neuroblasts, fibroblasts, glioblasts, germ cells, hepatocytes, chondrocytes, keratinocytes, smooth muscle cells, cardiac muscle cells, connective tissue cells, glial cells, epithelial cells, endothelial cells, hormone-secreting cells, cells of the immune system, and neurons. In one aspect, bone cells such as osteoclasts, osteocytes, and osteoblasts can be cultured with the coated substrates produced herein. Cells useful herein can be cultured in vitro, derived from a natural source, genetically engineered, or produced by any other means. Any source of cells can be used. Atypical or abnormal cells such as tumor cells can also be used herein. Cells that have been genetically engineered can also be used. Engineering involves programming the cell to express one or more genes, repressing the expression of one or more genes, or both. Genetic engineering can involve, for example, adding or removing genetic material to or from a cell, altering existing genetic material, or both. Embodiments in which cells are transfected or otherwise engineered to express a gene can use transiently or permanently transfected genes, or both. Gene sequences may be full or partial length, cloned or naturally occurring.

In the examples presented here, H1 (or WA01, or GE01) cells are used. However, it is contemplated that any embryonic stem cells or hESC may exhibit preferable characteristics when cultured on embodiments of the cell culture surfaces of the present invention.

In embodiments, monomers may be combined with additional monomers. In addition, monomers, either alone or mixed with additional monomers may be treated to induce polymerization of monomers into polymers or polymeric material or polymeric blends. Many methods are known in the art for inducing polymerization, including chemical polymerization and UV polymerization. In embodiments, the monomers may be mixed with a photo-initiator composition and exposed to UV light. And, in embodiments of the present invention, to ensure uniform coating of the substrate, monomers in solution may be diluted in an appropriate organic solvent such as, for example, ethanol. In embodiments, the solvent may be, for example, ethanol. Ethanol can be removed under slight vacuum or room temperature. The choice of solvent may be very important. For example, acetone, THF (tetrahydrofuran), DCM (dichloromethane may physically interact with plastic or polymeric substrates and interfere with the long term viability of a cell culture surface. Other solvents, such as DMF (Dimethylformamide), DMSO (Dimethylsulfoxide), and Acetonitrile are all high boiling point solvents that may require high temperature or high vacuum for evaporation and are not excluded but not preferred solvents for this process. The ethanol solvent may be, for example, a solvent having greater than about 75% ethanol. For example, an ethanol solvent may contain greater than 80%, greater than 90%, greater than 95%, greater than 97%, or greater than 99% ethanol. In various embodiments, the ethanol solvent consists essentially of ethanol. In some embodiments, an ethanol solvent consists essentially of ethanol and water. Polymerized monomers, or polymeric blends, may be applied to the substrate. In embodiments, combinations of two, three or four monomers may be polymeric blends and may be polymerized and applied to a substrate, or mixtures of two to ten or two to twenty monomers may be polymerized and applied to a surface or substrate. For example, monomers may be combined with additional monomers to provide single, bi- or trifunctional mixtures of monomers, and polymerized to form polymeric blends.

Monomers which have more than one active moiety, in this case (meth)acrylate moieties, are cross-linking monomers. The higher the percentage of cross-linking monomers in a mixture, the more cross-linked the cell culture surface will be. More cross-linked surfaces are harder surfaces. These hard surfaces are less likely to absorb water. If they are charged monomers, they may provide good wetability, and therefore high measured modulus while at the same time, these surfaces may be hard, non-porous surfaces. Highly crosslinked surfaces are not hydrogels. That is, they do not absorb liquid. These surfaces because of their physical properties outlined may also adsorb small and large bio-molecules present in the cell culture media and or proteins produced during cell growth which may further enhance growth and proliferation of cells including human embryonic stem cells on the surface.

Surfaces for cell culture can be described according to their characteristics such as hydrophobicity, hydrophilicity, surface charge or surface energy, wettability or contact angle, topography, modulus which describes the surface's stiffness versus compliance, degree of cross-linking of polymers, as well as chemical characteristics such as the surface expression of active chemical moieties such as oxygen or nitrogen.

Tables 1-6 show monomers and combinations of monomers which provide cell culture surfaces in embodiments of the present invention. The combinations of monomers shown in Table 1 provide more hydrophobic cell culture surfaces. Table 2 shows combinations of monomers which provide more hydrophilic cell culture surfaces. Table 3 shows neutral or positively charged (meth)acrylate surfaces. Table 4 shows negatively charged (meth)acrylate surfaces. Table 5 shows combinations of four monomers to yield a hydrophilic surface. Table 6 shows a (meth)acrylate surface having a penta-acrylate crosslinker.

In general, the cell culture surfaces made from the combinations of monomers shown in Tables 1-6 were made by first mixing appropriate proportions of monomers as defined below, to a mixture including a photo-initiator and a solvent, applying the solution to a 96 well plate, distributing the (meth)acrylate monomer solution over the surface of the well, allowing the solvent to evaporate, and inducing cross-linking of the monomers using a UV light source. This method produces a polymeric network, but not an interpenetrating network. Methods for making the cell culture surfaces are described in Example 1.

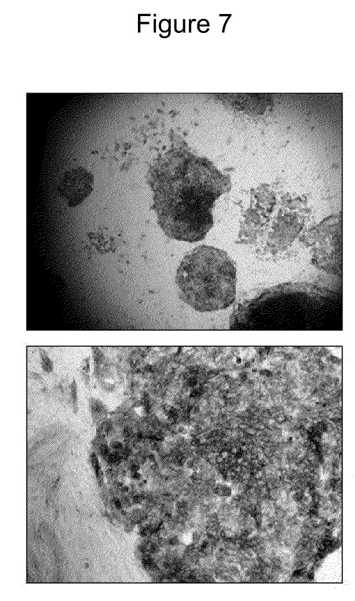

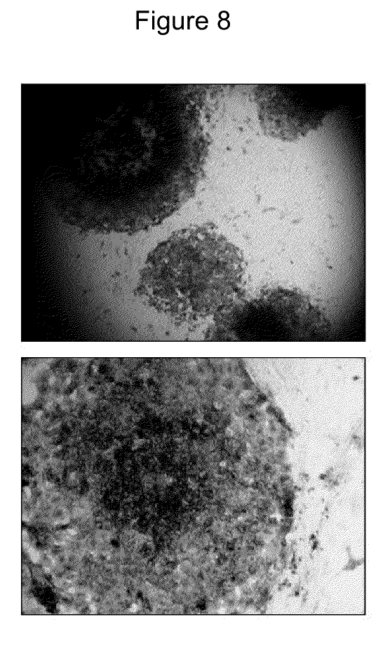

Surfaces were evaluated for their applicability as cell culture surfaces for undifferentiated hESCs, and assigned an R value using a rating system. The rating system takes into account the quantitative assessment of undifferentiated hESC attachment and growth on the surface with AttoPhos fluorescent assay as well as the quality of the cells on the surface, with BCIP/NBT staining. Alkaline phosphatase (AP) is a marker for undifferentiated embryonic stem cells. AP expression is lost or significantly reduced as cells differentiate. Cells grown on cell culture surface embodiments of the present invention were exposed to the alkaline phosphatase substrate, ATtoPhos, followed by measurement of fluorescent intensity resulted from the conversion of AttoPhos substrate into fluorescent product (described below in Example 3). BCIP/NBT and OCT3/4 staining were also performed. The BCIP/NBT assay is a colorimetric assay which also measures alkaline phosphatase. However, the BCIP/NBT substrate is converted into a purple precipitate if alkaline phosphatase is present, allowing for visual assessment of H1 hES cell colony morphology, as shown in FIG. 7 (H1 hES cells on Matrigel™) and FIG. 8 (H1 hESC cells on an embodiment of the (meth)acrylate surface of the present invention). Matrigel™ is a basement membrane preparation extracted from mouse sarcoma cells, available from BD Biosciences, Franklin Lakes, N.J., used as a positive control for undifferentiated hES cell surface.

Quality of cell growth, or the undifferentiated hESC attachment and proliferation on embodiments of (meth)acrylate surfaces of the present invention, was assessed by comparing the morphology of undifferentiated proliferating H1 hESC cells, including cell size, shape, and the interactions of one cell with another cell on the cell culture surfaces of the present invention with H1 hESC cells grown on Matrigel™. R values range from “A” surfaces, which support undifferentiated hES cell growth (80%+AttoPhos Fluorescence Intensity) and morphology similar to that exhibited by cells growing on Matrigel™ to “F” surfaces which were cytotoxic. “B” surfaces supported undifferentiated hES cell growth (80%+AttoPhos Fluorescence Intensity) similar to hESCs cultured on Matrigel™, but the hESC morphology was different from that exhibited by hESCs cultured on Matrigel “C” surfaces supported less undifferentiated hESC growth (50%-80% AttoPhos Intensity compared to Matrigel™). “D” surfaces did not exhibit satisfactory hESC attachment and growth (<50% AttoPhos Intensity compared to Matrigel™). Cytotoxicity of the surfaces was also determined using MRC5 cells. Surfaces which did not support MRC5 cell growth were considered toxic and were not tested against hESC cell growth.

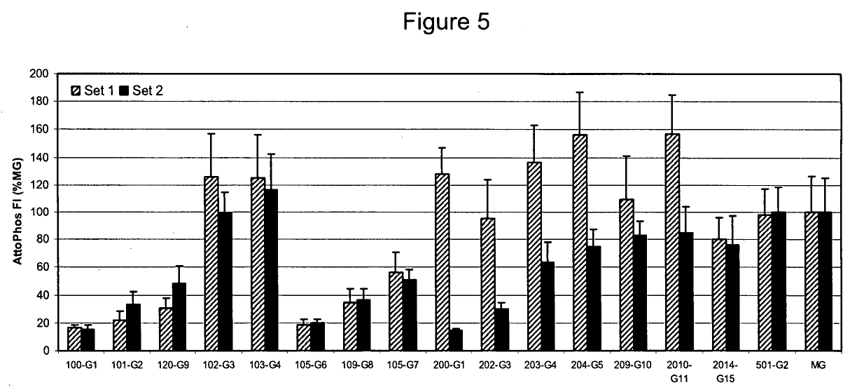

FIG. 5 shows AttoPhos fluorescence measurements taken from H1 hESC cells growing on embodiments of the cell culture surfaces of the present invention, normalized to measurements taken from H1 hES cells grown on Matrigel™. Fluorescence measurements indicate undifferentiated stem cell growth on that surface. As shown in FIG. 5, some of the hydrophilic cell culture surfaces were favorable for cell growth, as indicated by having fluorescence measurement of cell growth greater than or equal to control measurements from cells on Matrigel™. This information provides the quantitative measure of cell culture conditions which when combined with the qualitative measure, provides the cell culture rating (the R value).

TABLE 1
Hydrophobic (meth)acrylate Surfaces

| ID | Monomer 1 | Monomer 2 | Monomer 3 | R |
|---|---|---|---|---|
| 100G1 | 20% Stearyl acrylate 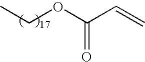 | 40% 1,6 hexandiol Diacrylate 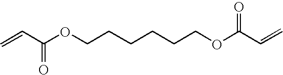 | 40% Trimethylpropane triacrylate 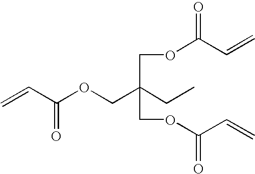 | D |
| 100G2 | 50% Stearyl acrylate 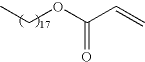 | 35% 1,6 hexanediol Diacrylate 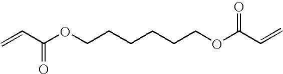 | 15% Trimethylpropane triacrylate 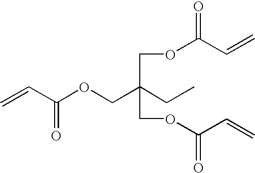 | * |
| 101G2 | 40% Lauryl acrylate 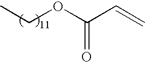 | 40% 1,6 hexandiol Diacrylate 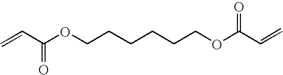 | 20% Trimethylpropane triacrylate 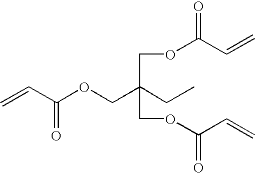 | D |
| 101G3 | 70% Lauryl acrylate 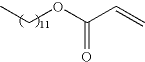 | 20% 1,6 hexandiol Diacrylate 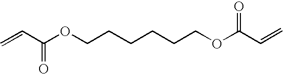 | 10% Trimethylpropane triacrylate 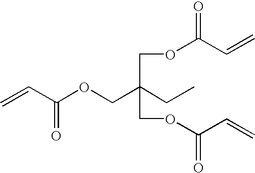 | * |
| 105G7 | 60% Dicyclopentadienyl Methacrylate 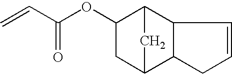 | 40% 1,6 Hexandiol Diacrylate 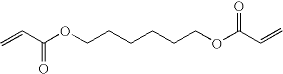 | — | D |
| 120G9 | 40% Lauryl (meth)acrylate 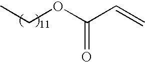 | 40% 1,6 Hexandiol Di(meth)acrylate 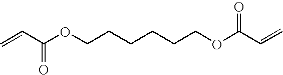 | 20% Trimethylpropane tri(meth)acrylate 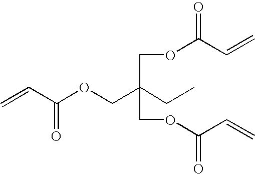 | D |

TABLE 2
Hydrophilic (meth)acrylate Surfaces

| ID | Monomer 1 | Monomer 2 | Monomer 3 | R |
|---|---|---|---|---|
| 102G3 | 17% Tris(2-hydroxy- ethyl) isocyanurate Triacrylate 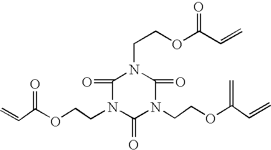 | 50% 1,6 hexanediol Diacrylate 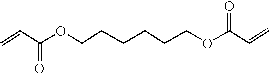 | 33% Trimethylpropane triacrylate 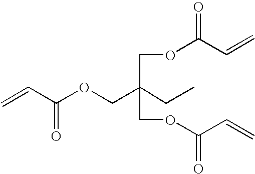 | B |
| 103G4 | 20% Tris(2-hydroxy Ethyl) isocyanurate Triacryalte 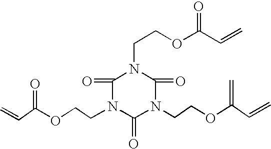 | 40% 1,6 hexanediol Diacrylate 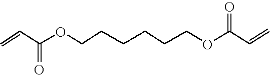 | 40% Trimethylpropane triacrylate 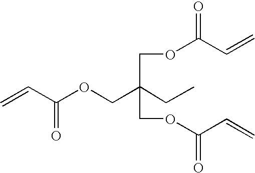 | B |
| 103G5 | 45% Tris(2-hydroxy Ethyl) isocyanurate Triacryalte 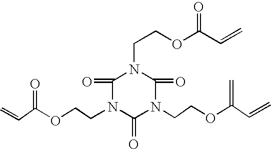 | 10% 1,6 hexanediol Diacrylate 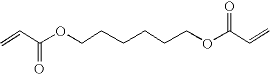 | 45% Trimethylpropane triacrylate 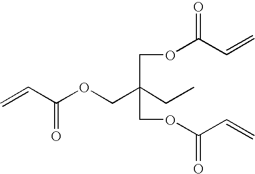 | * |
| 105G6 | 40% Caprolactone acrylate 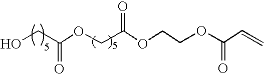 | 60% 1,6 Hexandiol Diacrylate 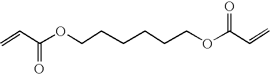 | — | D |
| 109G8 | 20% Caprolactone acrylate 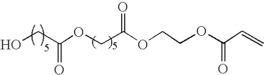 | 80% 1,6 Hexandiol Diacrylate 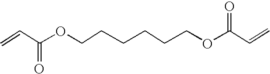 | — | D |
| 200G1 | 40% 2(2-Ethoxyethoxy) Ethyl acrylate 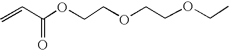 | 40% Tetrahydrofurfuryl acrylate 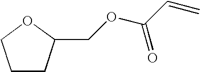 | 20% Proxylated Triglycerol Triacrylate 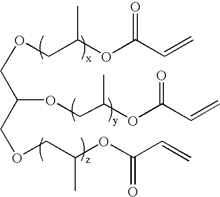 | D |
| 202G3 | 60% Proxylated Triglycerol Triacrylate 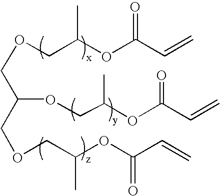 | 20% N-Vinyl-2-Pyrrolidone Methacrylate 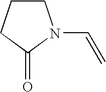 | 20% N,N-Dimethyl-Acrylamide 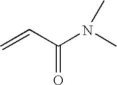 | D |
| 203G4 | 70% Tetrahydrofurfuryl acrylate 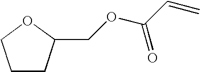 | 10% N-Vinyl-2-Pyrrolidone Methacrylate 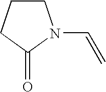 | 20% Tripropyleneglycol Diacrylate 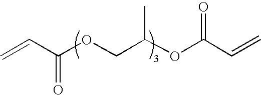 | B |
| 204G5 | 60% Proxylated Triglycerol Triacrylate 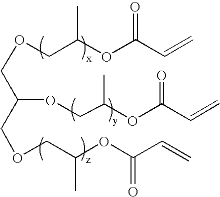 | 20% N-Vinyl-2-Pyrrolidone Methacrylate 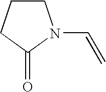 | 20% Tripropyleneglycol Diacrylate 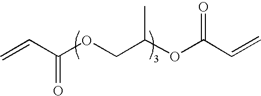 | B |
| 209G10 | 30% 2-N-Morpholinoethyl Methacrylate 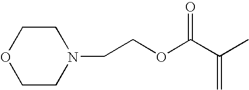 | 70% Tripropyleneglycol Diacrylate 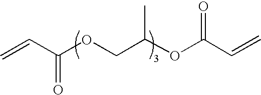 | — | B |

TABLE 3
Neutral or Positively Charged (meth)acrylate Surfaces

| | | | | |
|---|---|---|---|---|
| 4000-3 | 60% Tetraethylene Glycol Dimethacrylate 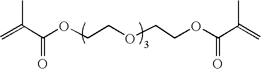 | 20% 1,4-Butanediol Dimethacrylate 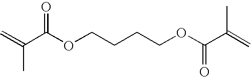 | 20% 2-(2-oxo-1-imidazolidinyl) methacrylate | B |
| 2000-4 | 80% 501G2 | 20% 2-N-Morpholino Ethyl Methacrylate 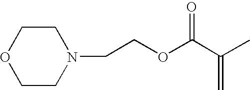 | | B |
| 2000-7 | 80% 501G2 | 20% 1-vinyl imidazole 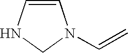 | | B |
| 4000-10 | 60% Tetraethylene Glycol Dimethacrylate 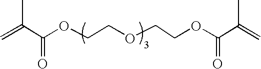 | 20% 1,4-Butanediol Dimethacrylate 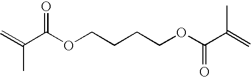 | 20% Poly(propylene)glycol (400) dimethacrylate 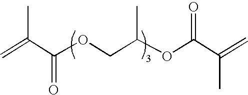 | C |

TABLE 4
Negatively Charged (meth)acrylate Surfaces

| | | | | |
|---|---|---|---|---|
| 4000-5 | 20% 2(Dimethyl amino) Ethyl methacrylate 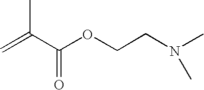 | 20% 1,4-Butanediol Dimethacrylate 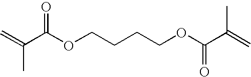 | 60% Tetraethylene Glycol Dimethacrylate 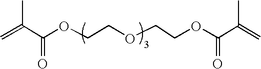 | B |
| 4000-13 | Pentaerythritol triacrylate 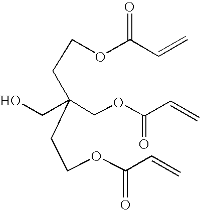 | 20% 1,4 Butanediol Dimethyacrylate 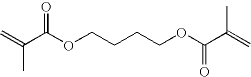 | 60% Tetraethylene Glycol Dimethacrylate 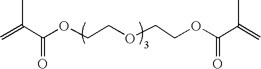 | B |
| 4000-16 | 20% Caprolactone acrylate 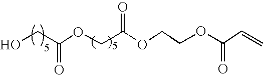 | 20% 1,4-Butanediol Dimethacrylate 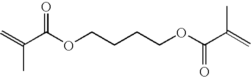 | 60% Tetraethylene Glycol Dimethacrylate 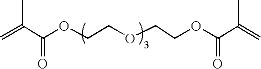 | B |
| 5000-2 | 60% Tripropylene Glycol Diacrylate 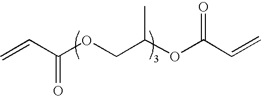 | 20% 1,5-Pentanediol dimethacrylate 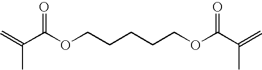 | 20% Tetrahydrofurfuryl acrylate 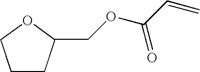 | B |
| 5000-16 | 60% Tripropylene Glycol Diacrylate 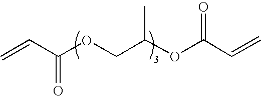 | 20% 1,5-Pentanediol dimethacrylate 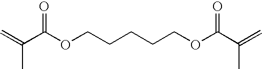 | 20% 2-(2-ethoxyethoxyl) ethyl acrylate 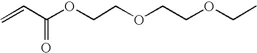 | B |
| 5000-17 | 60% Tripropylene Glycol Diacrylate 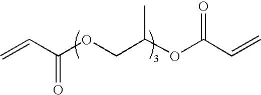 | 20% 1,5-Pentanediol dimethacrylate 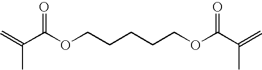 | 20% (t-butylamino) ethyl methacrylate 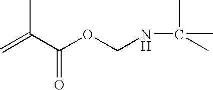 | B |
| 5000-25 | 60% Tripropylene Glycol Diacrylate 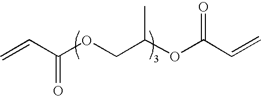 | 20% 1,5-Pentanediol dimethacrylate 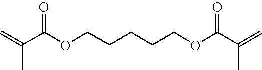 | 20% Lauryl methacrylate 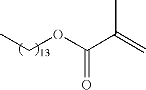 | B |
| 5000-27 | 60% Tripropylene Glycol Diacrylate 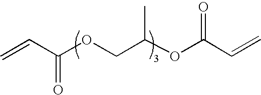 | 20% 1,5-Pentanediol dimethacrylate 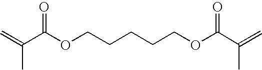 | 20% 501G2 | B |

TABLE 5
Other (meth)acrylate Surfaces with (1) to (2) Nitrogen Containing Ring Structures

| ID | Monomer 1 | Monomer 2 | Monomer 3 | Monomer 4 |
|---|---|---|---|---|
| 2010 G11 | 60% N-N- Dimethyl Acrylamide 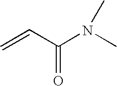 | 20% 2-(2-oxo- 1-Imidazolidinyl) Methacrylate 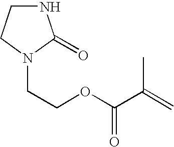 | 15% N-Vinyl-2- Pyrrolidone Methacrylate 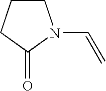 | 5% Pentaerythritol Triacrylate 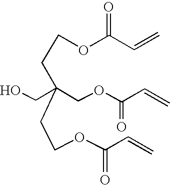 |
| 2014 G15 | 70% N-N- Dimethyl Acrylamide 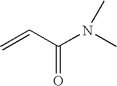 | 10% N-Vinyl-2- Pyrrolidone Methacrylate 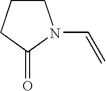 | 15% 2(2- Ethoxyethoxy) Methacrylate 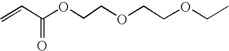 | 5% Pentaerythritol Triacrylate 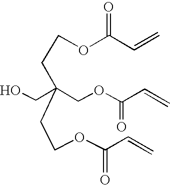 |

TABLE 6
Other (meth)acrylate surface (high crosslinked with high modulus)

| ID | Monomer 1 | Monomer 2 | R |
|---|---|---|---|
| 1013-2 | 70% Tripropylene glycol dimethacrylate 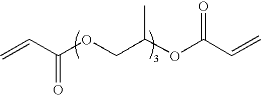 | 30% Dipenta-erythritol penta- acrylate 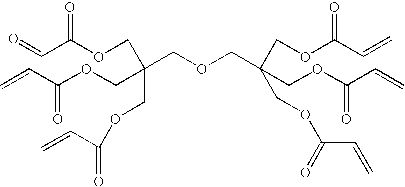 | B |

The compounds reported in Tables 1-6 are commercially available compounds, from sources such as Sartomer, Sigma-Aldrich and Polysciences. In Table 5, the ratings for 2010G11 and 2014G15 were “B.”

The monomers and mixtures of monomers illustrated in Table 1 are principally hydrophobic compounds. Hydrophobicity can be measured by contact angle. For example, compound 100G1, a mixture of 20% lauryl acrylate: 40% 1,6-hexanediol diacrylate: 20% trimethylpropane triacrylate yields a highly hydrophobic coating composition, with a contact angle of 92.1.

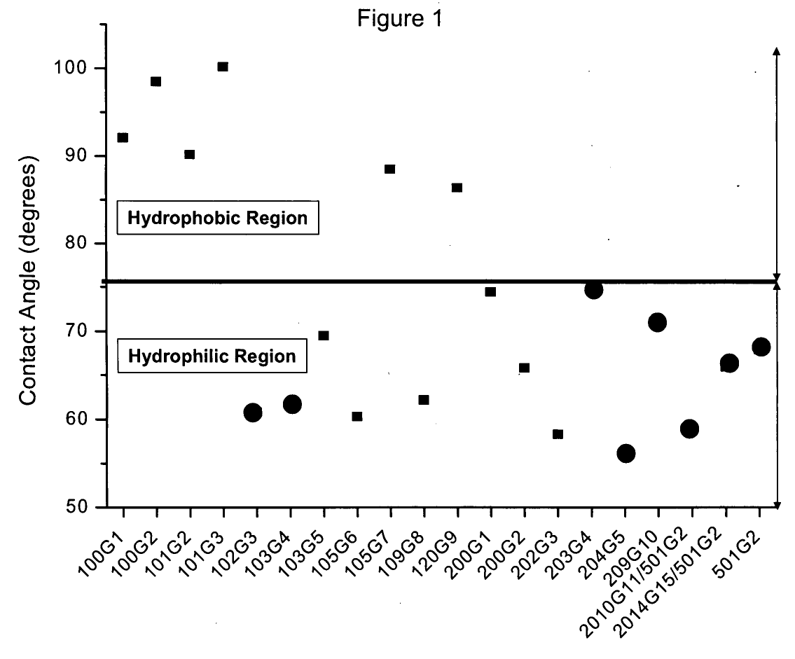

FIG. 1 is a graph showing the measured contact angle for embodiments of the compositions of the present invention. Meters and measuring devices are available from many suppliers to measure contact angles. These devices are available from, for example, KSV Instruments, Monroe, Conn., FDS Corp, Long Island, N.Y. and First Ten Angstroms, Portsmouth, Va. Contact angle is a measure of the angle at which a droplet of water sits on a surface. A droplet of water placed on a highly hydrophobic surface, a non-wettable surface, will form a tall, rounded droplet. The contact angle of such a drop will be high. On the other hand, a droplet of water placed on a highly hydrophilic surface, a wettable surface, will spread out and lay flat against the surface. The contact angle of a wettable surface will be low. As shown in FIG. 1, hydrophobic (meth)acrylates such as 100G1 will provide a surface having a high contact angle. The large circles in FIG. 1 represent surfaces that were rated “B” for hESC growth as measured after 48 hours in culture. More hydrophilic (or receding) (meth)acrylate compositions, including those listed in Table 2, are also shown in FIG. 1.

For the purposes of this analysis, an embodiment of the (meth)acrylate coatings of the present invention is considered hydrophobic (or advancing) if the measured contact angle is greater than about 85°, greater than about 80° or greater than about 76°. The contact angles of the compositions illustrated in FIG. 1, are reported in Tables 7 and 8.

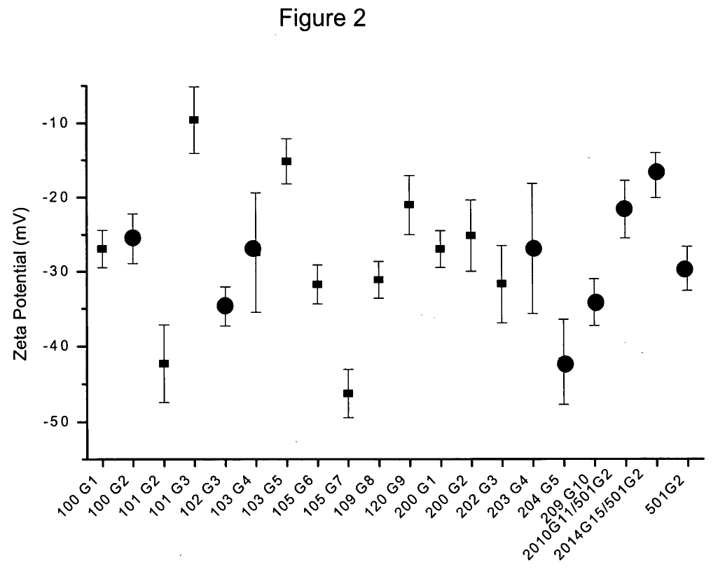

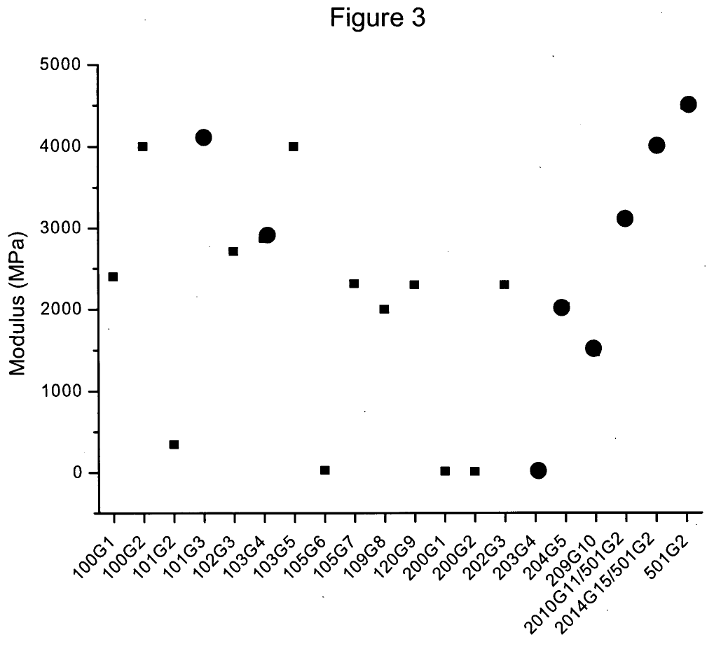

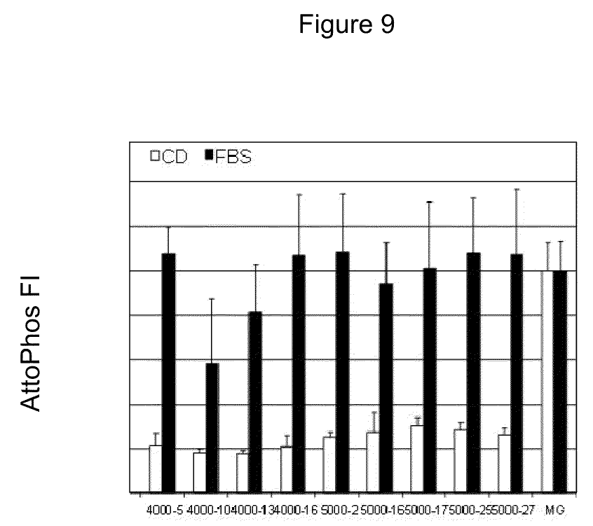

Charge, or Zeta potential (measured in mV) is illustrated in FIG. 2 and compositions having slightly positive or negative charge are shown in Tables 7 and 8. AttoPhos measurements taken from hESC cells cultured on embodiments of the coatings of the present invention, normalized to matrigel, are shown in FIG. 9. FIG. 9 shows that several of the negatively charged surfaces reported in Table 4 provide suitable cell culture coatings, for these cells, in the presence of serum. Modulus (measured in MPa) is illustrated in FIG. 3 and also reported in Tables 7 and 8.

TABLE 7
Contact Angle, Zeta Potential and Modulus for Hydrophobic
Surfaces

| | | Contact | Zeta | |
|---|---|---|---|---|
| | | Angle | Potential | Modulus |
| ID | Rating | (Deg.) | (mV) | (MPa) |
| 100G1 | D | 92.1 | −27.0 | 2400 |
| 100G2 | * | >90 | −25.6 | 4000 |
| 101G2 | D | 90.2 | −42.3 | 348 |
| 101G3 | * | >90 | −9.55 | 4100 |
| 105G7 | D | 88.5 | −46.2 | 2316 |
| 120G9 | D | 86.4 | −21.0 | 2300 |

The compositions shown in Table 1 are hydrophobic compositions. These hydrophobic compositions have contact angles greater than or equal to 86.4°. These hydrophobic compositions of (meth)acrylates did not provide surfaces that resulted in preferred growth conditions for undifferentiated hES cells in culture compared to Matrigel™ as indicated by the assigned rating, shown in Table 7. These surfaces had an “R” rating of D (the asterix * indicates that R ratings were not calculated for these surfaces).

In embodiments of the present invention, monomers and combinations of monomers which were more hydrophilic provided improved surfaces for cell growth. Table 8 shows contact angle measurements, along with measurements of Zeta potential and Modulus for embodiments of (meth)acrylate cell surfaces of the present invention which are considered to be hydrophilic (or receding), as defined by contact angles less than or equal to 76°.

TABLE 8
Contact Angle, Zeta Potential and Modulus for Hydrophilic
Surfaces

| | | Contact | Zeta | |
|---|---|---|---|---|
| | | Angle | Potenti | Modulus |
| ID | Rating | (Deg.) | (mV) | (MPa) |
| 102G3 | B | 60.8 | −34.6 | 2710 |
| 103G4 | B | 61.7 | −27.4 | 2872 |
| 103G5 | * | 69.5 | −15.1 | 4000 |
| 105G6 | D | 60.3 | −31.7 | 26 |
| 109G8 | D | 62.2 | −31.1 | 2000 |
| 200G1 | D | 74.4 | −27.0 | 16 |
| 200G2 | * | 65.8 | −25.1 | 12 |
| 202G3 | D | 58.3 | −31.7 | 2300 |
| 203G4 | B | 75.1 | −26.8 | 15.7 |
| 204G5 | B | 56.2 | −42.0 | 2029 |
| 209G10 | B | 70.7 | −34.1 | 1472 |
| 2010G11 | B | 58.7 | −21.6 | 3100 |
| (80%) + | | | | |
| 501G2 | | | | |
| (20%) | | | | |
| 2014G15 | B | 65.9 | −17.0 | 4000 |
| (80%) + | | | | |
| 501G2 | | | | |
| (20%) | | | | |
| 501G2 | B | 67.9 | −29.6 | 4500 |

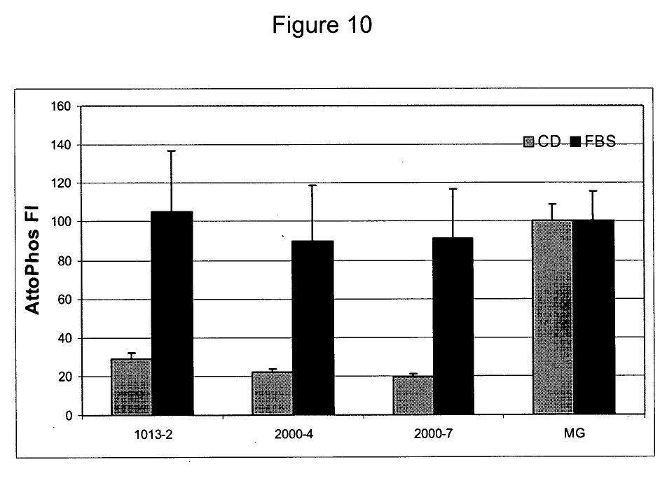

Zeta potential, or surface charge of a (meth)acrylate surface, can be measured and characterized. Cell culture surfaces may be charged or neutral, ionic or non-ionic, and may be cationic and/or zwitterionic. The zeta potential of monomer compositions listed in Table 1 and Table 2, as well as some mixtures of monomers containing mixture 501G2 shown in Table 5, are shown in FIG. 2. Large circles indicate surfaces which earned a rating of “B” after 48 hours of growth on the surfaces. No clear correlation between charge and favorable cell culture surface could be determined from the data shown. However, the negatively charged combinations reported in Table 4 were all “B” rated surfaces, as shown in FIG. 9. Neutral or slightly positively charged compositions shown in Table 3 were also mostly “B” rated surfaces, as shown in FIG. 10.

In addition to contact angle and zeta potential, these surfaces can be characterized by the modulus, or hardness, of the surface. FIG. 3 is a graph illustrating modulus measurements for surfaces shown in Tables 1 and 2 and some of the compositions in Table 5. The large circles shown in FIG. 3 indicate surfaces which were rated as “B” surfaces after 48 hours of growth, according to the rating system described above. In general, harder surfaces, surfaces with a modulus above 1400 MPa, provided surfaces which more successfully facilitated cell growth. Composition 1013-2, shown in Table 6, is a very hard surface, having a penta-acrylate, which provides 5 cross-linking groups. Although the modulus for this mixture was not measured, this mixture would fall within embodiments of the present invention characterized as “hard” surfaces. However, (meth)acrylate mixture 203G4 provided a B rated surface with a low modulus, or a softer surface. In embodiments, cell culture surfaces of the present invention have a modulus in the range of from 0 to 6000 MPa, or from 1000 to 5500 MPa. These surface that facilitate undifferentiated stem cell growth, although considered to be hydrophilic, are not considered hydrogels because they are more tightly crosslinked, displaying modulus ranges in the MPa and GPa range and do not absorb the quantity of water that hydrogels generally absorb. In addition, these surfaces are considered wettable but not necessarily swellable and are more wettable with a contact angle between 50 degrees −60 degrees, less wettable between 60 degrees to 70 degrees and least wettable between 70 degrees to 75 degrees. Hydrogels typically have modulus ranges in the kPa range, contact angles much less than 50 degrees and absorb 60-80% water in their structures.

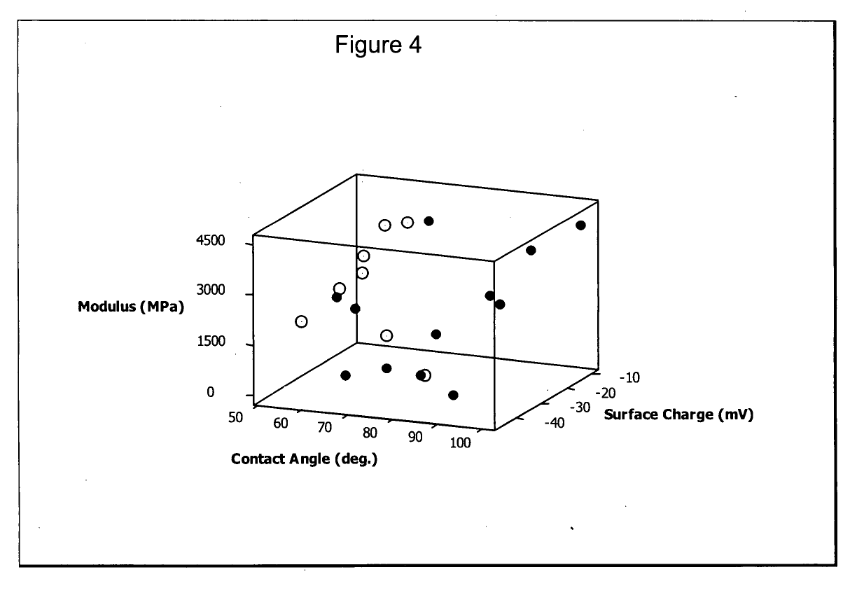

FIG. 4 is a three-dimensional plot of surfaces showing contact angle, surface charge, and modulus for the compositions listed in Tables 1, 2 and some of the compositions listed in Table 5. The open circles indicate “B” rated surfaces.

In embodiments, the cell culture surfaces of the present invention are highly cross-linked surfaces made from mixtures of monomer which are from 5 to 100% cross-linker monomers, from 10-100% cross-linker monomers, from 20%-100% cross-linker monomers or from 30%-100% cross-linker monomers. Cross-linker monomers are monomers having more than one active moiety, for example (meth)acrylate moieties.

When creating these mixtures for cell culture surfaces, and without being limited by a theory, one or more monomers in the mixture, according to embodiments of the present invention, provides the hydrophobicity or hydrophilicity of the surface. For example, one monomer listed in Tables 1 and 2 is either a hydrophobic or hydrophilic compound. For example, an (meth)acrylate compound made primarily from lauryl stearate will form a hydrophobic surface. That monomer may also be charged and exhibit a certain modulus when applied to a substrate. Polymers made only from the first monomer may be lightly, moderately or highly crosslinked, creating a surface which can also be described by the surface's hydrophobicity, charge and modulus.

In embodiments of the present invention, the addition of a second monomer in addition to the first monomer, polymerized or crosslinked, may add additional features or characteristics to the cell culture surface. The addition of a second monomer provides a bifunctional cross-linked polymer. The addition of the second monomer may provide additional chemical or physical characteristics which are desirable for the desired cell culture conditions. For example, a first monomer may be hydrophilic. A second monomer may have multiple (meth)acrylate groups. The number of (meth)acrylate groups may affect the hardness or softness of the cell culture surface. A third monomer, or more monomer or mixtures of monomers may also be added to provide trifunctional cross-linked polymer (and so on for the fourth monomer, or additional monomers, if appropriate). In additional embodiments of the present invention, the addition of a third or fourth monomer or additional monomer in the crosslinked or polymerized coating may add still additional characteristics to the cell culture surface. Additional monomers provide additional surface characteristics. For example, additional monomers may be adhesion promoters or may be multi-functional monomers to improve adhesion of the polymer layer to the substrate and reduce swelling in the polymer layer when the coated cell culture surface is exposed to the aqueous cell culture media, or provide any of the characteristics described above.

The following examples are included to demonstrate embodiments of the invention and are not intended to limit the scope of the invention in any way. It should be appreciated by those of skill in the art that the techniques disclosed in the examples which follow represent techniques discovered by the inventors to function well in the practice of the invention. However, those of skill in the art should, in light of the present disclosure, appreciate that many changes can be made in the specific embodiments which are disclosed and still obtain a like or similar result without departing from the spirit and scope of the invention

### EXAMPLE 1

### Preparation of Surfaces

### A. Preparation of (Meth)Acrylates

In a fume hood, using filtered light (cut off wavelength <460 nm), a photoinitiator stock solution was prepared by dissolving 1 wt % of photoinitiator i.e. Bis (2,4,6-trimethylbenzoyl)-phenylphosphine oxide (Irgacure 819, a light yellow powder photo-initiator with melting point of 127-133° C., specific gravity of 1.2, and absorption spectra with wavelength ranging from 340 to 440 nm, available from Ciba specialty chemicals, Tarrytown, N.Y.) in ethanol (200 proof) in a large bottle (500 or 1000 ml). (The concentration, volume and the type of photo-initiator may vary depending on experimental design). An appropriate weight (to a total of 10 g) of each (meth)acrylate monomer was placed in a glass vial (30 mL). A disposable dropper was used to transfer the liquid monomer into each vial. For highly viscous monomers, use an oven (at ˜60° C.) to warm up the monomers before transferring.

10 ml of photo-initiator stock solution was added into each glass vial containing the monomer(s) to make 1:1 (w/v) mixture and shaken to ensure that the monomer and photo-initiator solutions were thoroughly mixed. This preparation was the stock solution for that particular monomer. Other monomer/photo-initiator stock solutions were made in this similar manner.

Monomer/photo-initiator stock solutions were combined to create the mixtures of monomers as described in Tables 1-3. A total of 100 μl of monomer stock solution was dispensed into strips of polypropylene cluster tubes containing 8 wells, which can hold 8 different monomer blend formulations and placed on a rack.

Several mL of 200-proof ethanol were poured into a reagent reservoir. Using a semi-automated 1250 μl 8-channel pipetter, 400 μl of ethanol was transferred from the reservoir into each well of tube clusters filled with monomer/photo-initiator solution to further dilute the monomer to 1:9 with ethanol i.e. 10% monomer/photo-initiator solution: 90% ethanol and the cluster tubes were capped, inverted to mix and shaken on a microplate shaker on medium speed for 1 minute. These blends were then ready for filling into a 96 well plate.

### B. Application of Solutions to Surfaces

Plasma-treated cyclic olefin copolymer (TOPAS®) 96 well plates were filled with (meth)acrylate solution using an Automated Microplate Pippeting System from Biotek, Winooski, Vt., following the instructions provided by the manufacturer. The instrument is designed to fill two microplates at a time. Plates were placed on trays in a hood for at least three hours until the ethanol evaporated.

After the solvent was evaporated, plates were cured using pulsed UV light using a Xenon Model RC-800 from Xenon Corporation, Wilmington, Mass., according to the manufacturer's instructions setting the instrument to “high voltage” and “timed start.” Plates were purged with nitrogen for 60 seconds and exposed to UV light for 60 seconds.

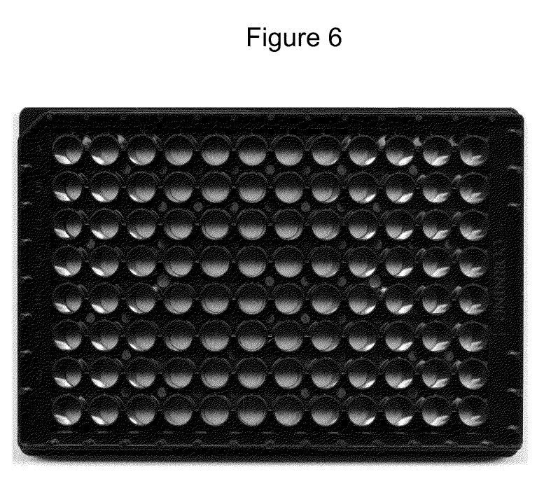

After curing, the wells of the plates were filled with ethanol (200 μl) and agitated on a shaker table at medium speed for 1 hour. Ethanol was discarded and the plates were washed with 400 μl pure water using a Tecan Power Washer 384 from Tecan AG, Switzerland. Water was removed and 200 μl of water was dispensed into each well, and the plates were allowed to incubate overnight at 40-42° C. Plates were then washed with 400 μl of extra pure water. Water was removed and plates were inverted and allowed to dry for 2-3 days in a vacuum oven at room temperature. The resulting cell culture coating was uniform when analyzed by microscopy. Plates were then sterilized by gamma sterilization. FIG. 6 is a photograph of a 96 well plate, coated with embodiments of the (meth)acrylate cell culture surface of the present invention.

### EXAMPLE 2

### Cytotoxicity Assay

Promega Corporation manufactures a cell proliferation assay kit (CellTiter 96® AQueous One Solution Cell Proliferation Assay7) that is specific for metabolically active cells. In this assay, a tetrazolium compound (3-(4,5-di methylthiazol-2-yl)-5-(3-carboxymethoxyphenyl)-2-(4-sulfophenyl)-2H-tetrazolium; MTS) is reduced by dehydrogenase enzymes in viable cells, resulting in a soluble colored formazan product that can be quantified by absorbance at 490 nm. The amount of formazan product, therefore, is directly related to the number of viable cells.

Human fibroblast cell line (fetal lung, MRC5) was selected for this study. Fibroblasts are ubiquitous cells and constitute a large percentage of stromal tissue in the human body. Commonly, MRC5 cells are used in cytotoxicity-based assays for their robustness compared to more advanced cell types; they have a characteristic morphology and a consistent pattern of attachment and proliferation that is easily noticed when disrupted (e.g. as a result of toxic compounds).

MRC5 cells were harvested using 0.05% trypsin/EDTA and seeded at a density of 15,000 cells/well. Cells were grown at standard cell culture conditions on embodiments of (meth)acrylate surfaces of the present invention for 72 hours. The CellTiter 96® AQueous One Solution Cell Proliferation Assay (G3581, Promega Corporation) was used to determine the relative number of viable cells on each surface after 72 hours in culture. The assay was performed according to the manufacturer's protocol. After aspiration of culture media, a 1:5 dilution of MTS tetrazolium reagent in phosphate buffered saline was added directly to cells. After 1 hour of incubation at 37° C. and 5% CO2, the absorbance at 490 nm was recorded. A surface was considered non-toxic if the Abs 490 nm was at least 80% of that for the positive control (Border CB-TOP/PtT=plasma-treated TOPAS); a surface was deemed toxic if the Abs 490 nm was 25% or less of that for PtT. All toxic surfaces were eliminated prior to hESC screening.

### EXAMPLE 3

### Cell Culture

A. Stock Culture of hESC Cell

H1 hES cells were cultured on Matrigel-coated TCT flasks in chemically defined culture medium (X-Vivo-10, 80 ng/ml hbFGF, 0.5 ng/ml hTGF-β1). Cells were passaged every 5-6 days at the seeding density of 5×10⁶ cells/T-75. For the experiments, cells were seeded at a density of 33,000 cells/well on Matrigel-coated or (meth)acrylate-coated 96-well plates using MultidropCombi (ThermoFisher) automated dispenser and cultured for 48 hrs in the same culture medium supplemented with 20% fetal bovine serum (FBS).

### EXAMPLE 4

### Alkaline Phosphate Screening Assays

Three different assays were used to screen H1 hESC growth on acrylic surfaces. First, AttoPhos quantitative assay was used to examine the number of alkaline phosphatase-positive (undifferentiated) hES cells within each well. Alkaline phosphatase (AP) is a marker for undifferentiated hES cells. AP expression is lost or significantly reduced as cells differentiate. BCIP/NBT staining was performed to assess H1 hES cell colony morphology compared to Matrigel™ (positive control). OCT3/4 is another pluripotency marker for undifferentiated hES cells. OCT3/4 immunofluorescence staining was also performed to further assess the undifferentiated status of H1 hES cells on embodiments of the present invention compared to Matrigel™.

### A. AttoPhos Screening Assay

For the AttoPhos screening assay, H1 hES cells were seeded at the density of 33,000 cells/well in 96-well plates coated with different acrylic formulations in the following culture medium: X-Vivo-10; 80 ng/ml hbFGF; 5 ng/ml hTGF-β1; 20% fetal bovine serum. Matrigel-coated wells in each plate were used as positive control. Cells were cultured at 37° C. with 5% CO₂ for 48 hrs. At the end of incubation time, cells were rinsed with 150 μl of Dulbecco's phosphate buffered saline (DPBS) and fixed with 4% paraformaldehyde for 10 min at R/T (70 μl/well of 96-well plate). Cells were washed once with 150 μl of DPBS, and treated for 10 min with 100 μl of AttoPhos fluorescent substrate (diluted 1:3 in DPBS) protected from light. AttoPhos fluorescent intensity at 485/535 nm was obtained using Victor 3 microplate reader (Perklin Elmer). AttoPhos fluorescent intensity for (meth)acrylic surfaces was normalized to that of Matrigel™ (see FIGS. 5, 9 and 10).

### B. BCIP/NPT Staining for Colony Morphology Assessment

After obtaining AttoPhos fluorescent intensity readings, cells were washed with 150 μl DPBS and processed for BCIP staining to assess cell colony morphology. Seventy μl of BCIP/NBT was added to each well and incubated for 20-30 min (to achieve desirable color intensity) at R/T with a mild agitation. BCIP/NBT staining system is based on the hydrolysis of BCIP and reduction of NBT producing a deep purple reaction product and stain. These reagents are available from several manufacturers including Abcamreagents, Cambridge, UK, and BioFX Laboratories, Owings Mills, Md. At the end of the staining, cells were washed once with 150 μl DPBS and either scanned or analyzed with light microscopy (see FIG. 2 as an example). H1 hES colony morphology on acrylic surfaces was compared to the colony morphology on Matrigel™ (positive control). FIG. 7 shows BCIP staining for cells grown on Matrigel™ (96 hours). FIG. 8 shows BCIP staining for cells grown on a “B” rated (meth)acrylate surface (96 hours). Note the similar cell morphology and colony formation.

### C. OCT3/4 Screening Assay

H1 hESC were plated in 96-well plates coated with embodiments of the (meth)acrylate cell culture surface or Matrigel™ in FBS supplemented medium. At the end of the incubation time, cell culture medium was aspirated and cells were washed once with DPBS followed by fixation with 70 μl of 4% PFA for 10 min at R/T. After washing with DPBS, cells were permeablized with 100% EtOH for 2 min (R/T), blocked with 10% heat-inactivated (HI) FBS in DPBS for 1 hr (R/T), followed by three washes with 100 μl DPBS, and treatment with Oct3/4 primary Ab (1 μg/ml in 2% HI FBS in DPBS), or the corresponding isotype control (goat IgG) for 1 hr at R/T. Cells were then washed three times with 100 μl DPBS followed by incubation with corresponding secondary Ab (DAG-AF568 at 1:1000 dilution in 2% Hi FBS in DPBS) plus Hoechst nuclear stain for 30 min at 37° C. protected from light. Finally, cells were washed three times with DPBS and examined with fluorescent microscopy or stored at 4° C. (data not shown).

### EXAMPLE 5

### Surface Rating System

H1 hES cells grown on surfaces of the present invention were stained and examined for AttoPhos fluorescent intensity and colony morphology and rated according to the following criteria: “A” surface: AttoPhos fluorescent intensity within 80-100% of Matrigel and similar to Matrigel colony morphology; “B” surface: AttoPhos fluorescent intensity within 80-100% of Matrigel but colony morphology is distinct from Matrigel; “C” surface: AttoPhos fluorescent intensity within 50-80% of Matrigel; “D” surface: AttoPhos fluorescent intensity below 50% of Matrigel. Additional cytotoxicity assays were performed. “F” surfaces were cytotoxic surfaces.

The invention being thus described, it would be obvious that the same may be varied in many ways by one of ordinary skill in the art having had the benefit of the present disclosure. Such variations are not regarded as a departure from the spirit and scope of the invention, and such modifications as would be obvious to one skilled in the art are intended to be included within the scope of the following claims and their legal equivalents.

We claim:

1. A composition for making cell culture surfaces comprising a mixture of at least two UV-curable monomers wherein one of the at least two monomers is selected from the group consisting of tris(2-hydroxy-ethyl) isocyanurate triacrylate, tetrahydrofurfuryl acrylate, proxylated triglycerol triacrylate, 2-N-morpholinoethyl methacrylate, bis(2-methacryoyloxyethyl) N,N′-1,9-nonylene biscarmate diurethane dimethacrylate, 2-(2-oxo-1-imidazolidinyl) methacrylate 1-vinyl imidazole, N-vinyl-2-pyrrolidone methacrylate, pentaerythritol triacrylate, N—N-dimethyl acrylamide, stearyl acrylate, lauryl acrylate, lauryl methacrylate, dicyclopentadienyl methacrylate, caprolactone acrylate, and 2(2-ethoxyethoxy) ethylacrylate, dipentaerythritol penta-acrylate, 2 (dimethyl amino) ethyl methacrylate, pentaerythritol tri-acrylate, and 2-(t-butylamino)ethyl methacrylate.

2. The composition of claim 1 wherein one of the at least two UV-curable (meth)acrylate monomers is selected from the group consisting of tris(2-hydroxy-ethyl) isocyanurate triacrylate, tetrahydrofurfuryl acrylate, proxylated triglycerol triacrylate, 2-N-morpholinoethyl methacrylate, bis(2-methacryoyloxyethyl) N,N′-1,9-nonylene biscarmate diurethane dimethacrylate, 1-imidazolidinyl methacrylate, N-vinyl-2-pyrrolidone methacrylate, pentaerythritol triacrylate and N—N-dimethyl acrylamide.

3. The composition of claim 1 further comprising at least one UV-curable monomer selected from the group consisting of 1,6 hexanediol diacrylate, tetraethylene glycol dimethacrylate, tripropyleneglycol diacrylate, 1,4-butanediol diacrylate trimethylpropane triacrylate and 1,5 pentanediol dimethacrylate.

4. The composition of claim 1 further comprising at least two UV-curable monomers selected from the group consisting of 1,6 hexanediol diacrylate, tetraethylene glycol dimethacrylate, tripropyleneglycol diacrylate, 1,4-butanediol diacrylate trimethylpropane triacrylate and 1,5 pentanediol dimethacrylate.

5. The composition of claim 1 wherein at least 5% of the UV-curable monomer in the composition is cross-linking monomer.

6. The composition of claim 1 further comprising a solvent with the provision that the solvent is not dimethylformamide (DMF), dichloromethane (DCM) or tetrahydrofuran (TH F).

7. The composition of claim 6 wherein the solvent is volatile.

8. The composition of claim 6 wherein the solvent is ethanol.

9. The composition of claim 1 wherein the at least two UV curable monomer mixture comprises:
tris(2-hydroxy-ethyl) isocyanurate triacrylate, 1,6 hexanediol diacrylate and trimethylpropane triacrylate;
tetrahydrofurfuryl acrylate, N-vinyl-2-pyrrolidone methacrylate, and N—N-dimethyl acrylamide;
proxylated triglycerol triacrylate, N-vinyl-2-pyrrolidone methacrylate and tripropylene glycol diacrylate;
morpholinoethyl methacrylate and tripropylene glcycol diacryate;
bis(2-methacryoyloxyethyl) N,N′-1,9-nonylene biscarmate diurethane dimethacrylate, 1,4-butanediol diacrylate and N-hexyl acrylate;
bis(2-methacryoyloxyethyl) N,N′-1,9-nonylene biscarmate diurethane dimethacrylate, 1,4-butanediol diacrylate, N-hexyl acrylate and 2-N-morpholinoethyl methacrylate;
bis(2-methacryoyloxyethyl) N,N′-1,9-nonylene biscarmate diurethane dimethacrylate, 1,4-butanediol diacrylate, N-hexyl acrylate and 1-vinyl imidazole;
bis(2-methacryoyloxyethyl) N,N′-1,9-nonylene biscarmate diurethane dimethacrylate, 1,4-butanediol diacrylate and N-hexyl acrylate, tripropylene glycol diacrylate and 1,5-pentanediol dimethacrylate;
2-(2-oxo-1-imidazolidinyl) methacryalte, tetraethylene glycol dimethacrylate and 1,4-butanediol dimethacrylate;
polypropylene glycol (400) dimethacrylate, tetraethylene glycol dimethacrylate and 1,4-butanediol dimethacrylate;
2-dimethyl amino ethyl methacrylate, tetraethylene glycol dimethacrylate and 1,4-butanediol dimethacrylate;
pentaerythritol triacrylate, tetraethylene glycol dimethacrylate and 1,4-butanediol dimethacrylate;
tetraethylene glycol dimethacrylate, 1,4-butanediol dimethacrylate, and caprolactone acrylate;
caprolactone acrylate, tetraethylene glycol dimethacrylate and 1,4-butanediol dimethacrylate;
tetrahydrofurfuryl acrylate, tripropylene glycol diacrylate, and 1,5-pentanediol dimethacrylate;
2-(2-ethoxyethoxyl)ethyl acrylate, tripropylene glycol diacrylate, and 1,5-pentanediol dimethacrylate;
lauryl methacrylate, tripropylene glycol diacrylate, and 1,5-pentanediol dimethacrylate;
N—N-dimethyl acrylamide, 2-(2-oxo-1-imidazolidinyl) methacrylate, N-vinyl-2-pyrrolidone methacrylate, and pentaerythritol triacrylate; or,
Dimethyl acrylamide, pyrrolidone methacrylate, 2(2-ethoxyethoxy) methacrylate; pentaerythritol triacrylate; and,
Tripropylene glycol dimethacrylate and dipenta-erythritol penta-acrylate.

10. A cell culture article comprising a polymeric coating disposed on a surface of a cell culture vessel wherein the polymeric coating is formed from at least two (meth)acrylate monomers and wherein the resulting surface has a contact angle of less than 80° and a modulus of from 1000 to 5500 mPa.

11. The cell culture article of claim 10 wherein the polymeric coating comprises at least two (meth)acrylate monomers selected from the group consisting of tris(2-hydroxy-ethyl) isocyanurate triacrylate, tetrahydrofurfuryl acrylate, proxylated triglycerol triacrylate, 2-N-morpholinoethyl methacrylate, bis(2-methacryoyloxyethyl) N,N′-1,9-nonylene biscarmate diurethane dimethacrylate, 2-(2-oxo-1-imidazolidinyl) methacrylate 1-vinyl imidazole, N-vinyl-2-pyrrolidone methacrylate, pentaerythritol triacrylate, N—N-dimethyl acrylamide, stearyl acrylate, lauryl acrylate, lauryl methacrylate, dicyclopentadienyl methacrylate, caprolactone acrylate, and 2(2-ethoxyethoxy) ethylacrylate, dipentaerythritol penta-acrylate, 2 (dimethyl amino) ethyl methacrylate, pentaerythritol tri-acrylate, and 2-(t-butylamino)ethyl methacrylate.

12. The cell culture article of claim 10 further comprising at least one UV-curable monomer selected from the group consisting of 1,6 hexanediol diacrylate, tetraethylene glycol dimethacrylate, tripropyleneglycol diacrylate, 1,4-butanediol diacrylate Trimethylpropane Triacrylate and 1,5 pentanediol dimethacrylate.

13. The cell culture article of claim 10 further comprising at least two UV-curable monomers selected from the group consisting of 1,6 hexanediol diacrylate, tetraethylene glycol dimethacrylate, tripropyleneglycol diacrylate, 1,4-butanediol diacrylate trimethylpropane triacrylate and 1,5 pentanediol dimethacrylate.

14. A method for preparing a cell culture surface comprising:
a. mixing UV-curable monomers together with a photopolymerizing agent in a solvent wherein the solvent is not DMF, DCM or THF;
b. applying the UV-curable monomer mixture to a cell culture substrate;
c. allowing the solvent to evaporate; and,
d. exposing the coated substrate to UV light;
e. wherein the UV curable monomer mixture comprises:
tris(2-hydroxy-ethyl) isocyanurate triacrylate, 1,6 hexanediol diacrylate and trimethylpropane triacrylate;
tetrahydrofurfuryl acrylate, N-vinyl-2-pyrrolidone methacrylate, and N—N-dimethyl acrylamide;
proxylated triglycerol triacrylate, N-vinyl-2-pyrrolidone methacrylate and tripropylene glycol diacrylate;
morpholinoethyl methacrylate and tripropylene glcycol diacryate;
bis(2-methacryoyloxyethyl) N,N′-1,9-nonylene biscarmate diurethane dimethacrylate, 1,4-butanediol diacrylate and N-hexyl acrylate;
bis(2-methacryoyloxyethyl) N,N′-1,9-nonylene biscarmate diurethane dimethacrylate, 1,4-butanediol diacrylate, N-hexyl acrylate and 2-N-morpholinoethyl methacrylate;
bis(2-methacryoyloxyethyl) N,N′-1,9-nonylene biscarmate diurethane dimethacrylate, 1,4-butanediol diacrylate, N-hexyl acrylate and 1-vinyl imidazole;
bis(2-methacryoyloxyethyl) N,N′-1,9-nonylene biscarmate diurethane dimethacrylate, 1,4-butanediol diacrylate and N-hexyl acrylate, tripropylene glycol diacrylate and 1,5-pentanediol dimethacrylate;
2-(2-oxo-1-imidazolidinyl) methacryalte, tetraethylene glycol dimethacrylate and 1,4-butanediol dimethacrylate;
polypropylene glycol (400) dimethacrylate, tetraethylene glycol dimethacrylate and 1,4-butanediol dimethacrylate;
2-dimethyl amino ethyl methacrylate, tetraethylene glycol dimethacrylate and 1,4-butanediol dimethacrylate;
pentaerythritol triacrylate, tetraethylene glycol dimethacrylate and 1,4-butanediol dimethacrylate;
tetraethylene glycol dimethacrylate, 1,4-butanediol dimethacrylate, and caprolactone acrylate;
caprolactone acrylate, tetraethylene glycol dimethacrylate and 1,4-butanediol dimethacrylate;
tetrahydrofurfuryl acrylate, tripropylene glycol diacrylate, and 1,5-pentanediol dimethacrylate;
2-(2-ethoxyethoxyl)ethyl acrylate, tripropylene glycol diacrylate, and 1,5-pentanediol dimethacrylate;
lauryl methacrylate, tripropylene glycol diacrylate, and 1,5-pentanediol dimethacrylate;
N—N-dimethyl acrylamide, 2-(2-oxo-1-imidazolidinyl) methacrylate, N-vinyl-2-pyrrolidone methacrylate, and pentaerythritol triacrylate;
dimethyl acrylamide, pyrrolidone methacrylate, 2(2-ethoxyethoxy) methacrylate; pentaerythritol triacrylate; or,
tripropylene glycol dimethacrylate and dipenta-erythritol penta-acrylate.

15. The method of claim 13 wherein the solvent is ethanol.

16. A system for cell culture comprising:
a cell culture vessel having a cell culture surface comprising a UV-cured mixture of at least two monomers wherein one of the at least two monomers is selected from the group consisting of tris(2-hydroxy-ethyl) isocyanurate triacrylate, tetrahydrofurfuryl acrylate, proxylated triglycerol triacrylate, 2-N-morpholinoethyl methacrylate, bis(2-methacryoyloxyethyl) N,N′-1,9-nonylene biscarmate diurethane dimethacrylate, 2-(2-oxo-1-imidazolidinyl) methacrylate 1-vinyl imidazole, N-vinyl-2-pyrrolidone methacrylate, pentaerythritol triacrylate, N—N-dimethyl acrylamide, stearyl acrylate, lauryl acrylate, lauryl methacrylate, dicyclopentadienyl methacrylate, caprolactone acrylate, and 2(2-ethoxyethoxy) ethylacrylate, dipentaerythritol penta-acrylate, 2 (dimethyl amino) ethyl methacrylate, pentaerythritol tri-acrylate, and 2-(t-butylamino)ethyl methacrylate.
i. chemically defined media, with at least 20% fetal bovine serum and
ii. embryonic stem cells.

17. The system of claim 15 wherein the cell culture surface further comprises a monomer selected from the group consisting of 1,6 hexanediol diacrylate, tetraethylene glycol dimethacrylate, tripropyleneglycol diacrylate, 1,4-butanediol diacrylate trimethylpropane triacrylate and 1,5 pentanediol dimethacrylate.

18. The system of claim 15 wherein the chemically defined media further comprises hbFGF and hTGF-β1.

19. The system of claim 15 wherein the embryonic stem cells are undifferentiated human embryonic stem cells.
